(19) United States
(12) Patent Application Publication
(10) Pub. No.: US 2018/0001001 A1
(43) Pub. Date: Jan. 4, 2018

(54) Novel hands-free breast pumping system

(71) Applicants: Mengju Wu, San Jose, CA (US); Banqiu Wu, San Jose, CA (US)

(72) Inventors: Mengju Wu, San Jose, CA (US); Banqiu Wu, San Jose, CA (US)

(21) Appl. No.: 15/201,452

(22) Filed: Jul. 3, 2016

(51) Int. Cl. A61M 1/06; A61M 39/10

(52) U.S. Cl. CPC A61M 1/064; A61M 39/10; A61M 2205/52; A61M 2205/50; A61M 2205/3337; A61M 2205/07; A61M 2209/088; A61M 2205/3344

(57) ABSTRACT

A hands-free breast pump system uses an advanced air bag for excellent contact between the air bag and breast so that a vacuum near the nipple can be guaranteed. The breast pumping and non-pumping condition can be easily converted by inflation and deflation of air bag using a computer-controlled microsystem. These properties not only make breast-pumping hands-free, but also allows the wearer to keep her clothing and bra on. The automatic control of breast milk expression also realizes hands-free breast milk pumping in public or during sleep. The control of pump time and duration can be programmed in a program or in an app. The breast milk expression system can be used independently, or together with a bra, or a shirt. The system disclosed in this patent can also simulate breast milk expression by a real baby by using a pulsing pressure in the air bag, which can squeeze the nipple periodically according a predetermined program.

### FIELD

The embodiment of the present invention is generally related to a breast milk pump system. More specifically, the present invention relates to a hands-free and automatic breast milk pump system.

### BACKGROUND

Breast pump systems for breast milk expression are well known and in high demand in market. The general methodology is to apply a vacuum to aid in breast milk expression combined with a funnel piece held against a woman's breast. The vacuum is applied to the nipples to express breast milk. The breast milk flows from a nipple through the funnel to a breast milk container.

Breast milk pumping systems currently available in-market use the method mentioned above. It requires a woman to remove her clothing and bra and takes about 20 to 30 minutes. It is a cumbersome and inconvenient process, and mother's rooms are not available in every company or public area.

Therefore, a hands-free breast pump system is proposed. However, for various reasons, the methods proposed have not gained popularity. In order to solve the technical problems, some patents have been filed.

U.S. Pat. No. 8,307,463 (Ritchie) disclosed a method for a hands-free pump and bra system. The method uses multiple layers of a bra and multiple elastic bands to maintain the vacuum around the nipple area. However, variation in the shape and size of breasts may cause vacuum leaking, and the tight elastic band may cause discomfort.

U.S. Pat. Nos. 9,167,855 and 8,323,070, as well as U.S. Pat. No. 8,192,247 (Abbaszadeh) disclosed a pumping/nursing bra that aims to make breast nursing and hands-free pumping easier. He also used an elastic band to ensure the effective contact with breast. However, a tight band causes discomfort, whereas a loose band will lose vacuum near nipple, resulting in breast pumping failure.

U.S. Pat. No. 8,801,495 (Guindon) invented an apparatus that integrated the bra and pump. The apparatus uses a cup held by a bra to cover the breast. The cup is held against the breast to seal a space for vacuum pumping. U.S. Pat. No. 6,227,936 (Mandoza) disclosed a hands-free pump differing from a traditional one. It includes a detachable cup for hands-free breast pumping. When not nursing, the cup is detached. U.S. Pat. No. 6,213,840 (Han) disclosed a hands-free breast pumping system. It uses a strong elastic material to hold the pumping system so pumping can be operated hands-free. For these inventions, the challenges are still the vacuum security. The tight contact between funnel and breast can secure the vacuum but is not comfortable for the wearer.

The most challenge technology for a hands-free breast pumping system is the maintaining of the vacuum around the nipple while maintaining comfort for the wearer. Current methods use a cup shape or funnel covering the breast while pushing against the breast using strong bands. If the elastic is not strong enough, the vacuum seal will break and breast milk expression will not work.

### SUMMARY

The principle object of this invention is to provide an advanced, easy-to-use, hands-free breast pump system that is fully operated with clothing on and is programmable. The new system also supports mobile device apps for programming and nighttime use during sleep. The key part of the system is the specially designed air bag seal device. The main advantage is its comfort when not pumping, and easy vacuum seal during breast milk expression. There is no need for any strong bands, only a slight hold. This advantage results from the vacuum space seal ensured by one side of air bag effectively making contact with the breast. Another advantage is the simulation of baby's sucking motion of breast milk. The air bag can be programmed to have a pulse pressure function, causing periodical suction of nipple.

This invention provides hands-free breast pumping with a very simple hookup without the need to remove the bra or clothing. When not in use, the parts of system on end users are only an air bag (with the air pumped out), a connector, a milk hose, and a hose coupling. These parts can be attached to bra or shirt without being detected.

The air bag is made of soft materials, such as silicone gel, silicon rubber, or thin plastics. With the inflation of the air bag, the air bag changes its surface shape to fit breasts of a variety of shapes. Due to the good contact between soft material and breast, very low pressure is applied by the air bag, so that there is no need to use a strong elastic band to hold it in place. The soft material used for air bag also makes the cup shape cover commonly used for hands-free pumping unnecessary because the excellent seal enabled by air bag material can ensure the vacuum buildup near the nipple area.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts one embodiment of hands-free breast pump system using a separate vacuum pump and air bag pump;

FIG. 2 depicts one embodiment of hands-free breast pump system using only one air pump for both pumping breast milk and inflation or deflation of the air bag, controlled by using an automatic control device;

FIG. 3 depicts one embodiment of hands-free breast pump system using only one air pump for both pumping breast milk and inflation of the air bag;

FIG. 4 depicts one embodiment of breast area setup of the hands-free breast pump system.

### DETAILED DESCRIPTION

Embodiments of the present invention generally provide apparatus for hands-free pumping of milk from the breasts. Particularly, embodiments of the present invention provide apparatus for pumping milk from breast with hands-free and programmable capability. In one embodiment, a pulsating suction is applied on nipple during breast pumping to simulate the motion of a baby sucking milk from his or her mother's breast.

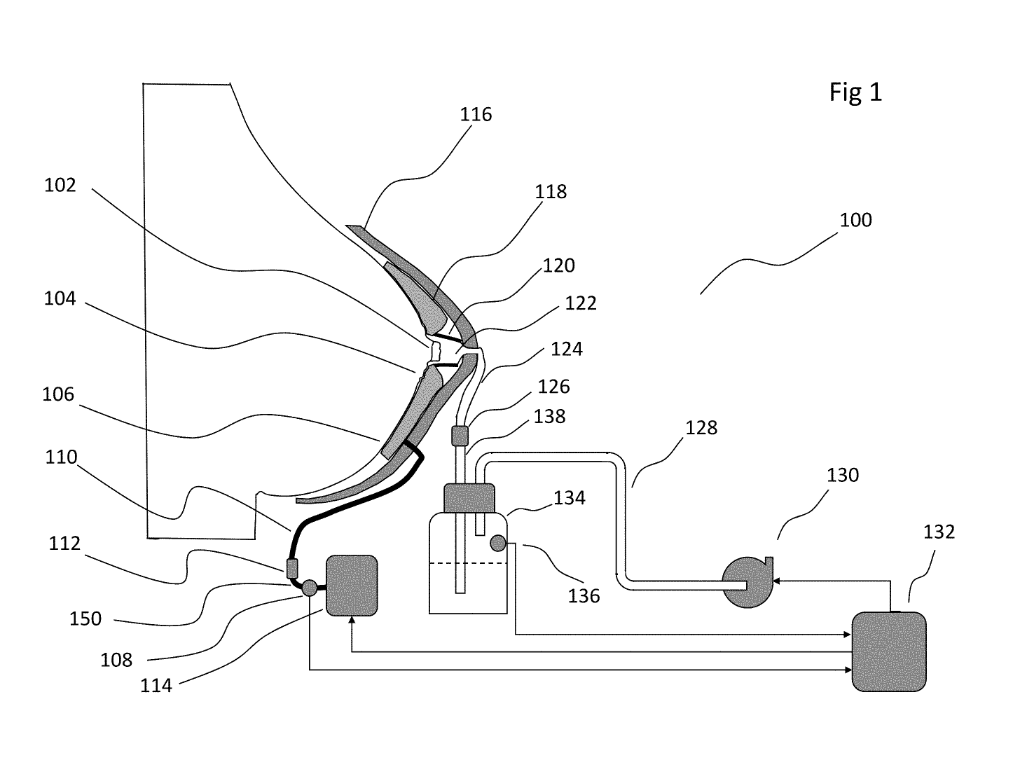

FIG. 1 schematically illustrates a hands-free breast pumping system 100 at the condition of in accordance with one embodiment of the present invention. The pumping system 100 generally comprises a vacuum space 122, air bag 118, and milk hose 124. In one embodiment, air bag 118 has a donut shape for securing vacuum in vacuum space 122.

The key to pumping breast milk is keeping a moderate vacuum around nipple area. In one embodiment, the vacuum space 122 is built by using air bag 118. By inflating air bag 118 using air bag pump 114, one surface of air bag 118 makes contact with the breast to seal vacuum space 122 effectively. The contact areas include areola 104 and its surrounding area on breast 106.

The inflation of air bag 118 is performed by supplying air from air bag pump 114 to air bag 118 through pump hose 150, air bag hose coupling 112, and air bag hose 110. In one embodiment, the air bag pump 114 is controlled by control system 132. The control system 132 controls the pressure in air bag 118 based on the measurement result from air bag pressure sensor 108 feeding back to control system 132.

For effective contact between air bag 118 and areola 104 and the surrounding area of areola 104 on breast 106, moderate surface area is ensured by design of air bag 118. Materials of air bag 118 play an important role in effectiveness of seal of vacuum space 122. In one embodiment, the material of air bag 118 is latex rubber. In another embodiment, the material of air bag 118 is silicon rubber, or silicone gel.

To maintain the stability of vacuum space 122, the connector 120 is configured to keep the contact area in correct position. In one embodiment, one end of the connector 120 is in contact with areola 104 or the side surface of nipple 102. The other end of the connector 120 connects to the milk hose 124.

FIG. 1 depicts that the air bag 118, connector 120, and milk hose 124 are fixed on bra 116. In one embodiment, air bag 118, connector 120, and milk hose 124 are held by a specially designed strip which is independent of bra 116.

When a baby sucks milk from the breast, a vacuum and periodical squeeze on nipple are applied. To simulate a baby's sucking of breast milk, in one embodiment, nipple 102 is periodically squeezed by connector 120 by using a pulse pressure in air bag 118 controlled by control system 132. To achieve a comfortable feeling for the wearer, in one embodiment, the material of connector 120 is silicone gel or silicon rubber.

Breast milk is sucked from nipple 102 through connector 120, milk hose 124, milk hose coupling 126, and bottle hose 138 to milk bottle 134. The milk pumping strength is determined by the vacuum pressure in milk bottle 134. In one embodiment, vacuum sensor 136 is located in milk bottle 134 for the purpose of control of the vacuum in vacuum space 122. The vacuum pressure data obtained by vacuum sensor 136 is input to control system 132. Control system 132 adjusts pump air flow rate in air hose 128 by vacuum pump 130 to control the vacuum in milk bottle 134.

In one embodiment, control system 132 comprises a central process unit (CPU), dynamic random-access memory (DRAM), non-volatile memory, analog/digital (A/D) converter, and digital/analog (D/A) converter. The control program is saved in non-volatile memory.

One of the important advantages of this invention is the programming capability. In one embodiment, the breast pumping schedule is controlled by a program stored in control system 132. This advantage is necessary for breast pump during sleep in the evening when the baby's mother is tired and wishes to have a good night's sleep.

In one embodiment, breast expression is controlled and programmed by using a cell phone with a pre-programmed mobile app.

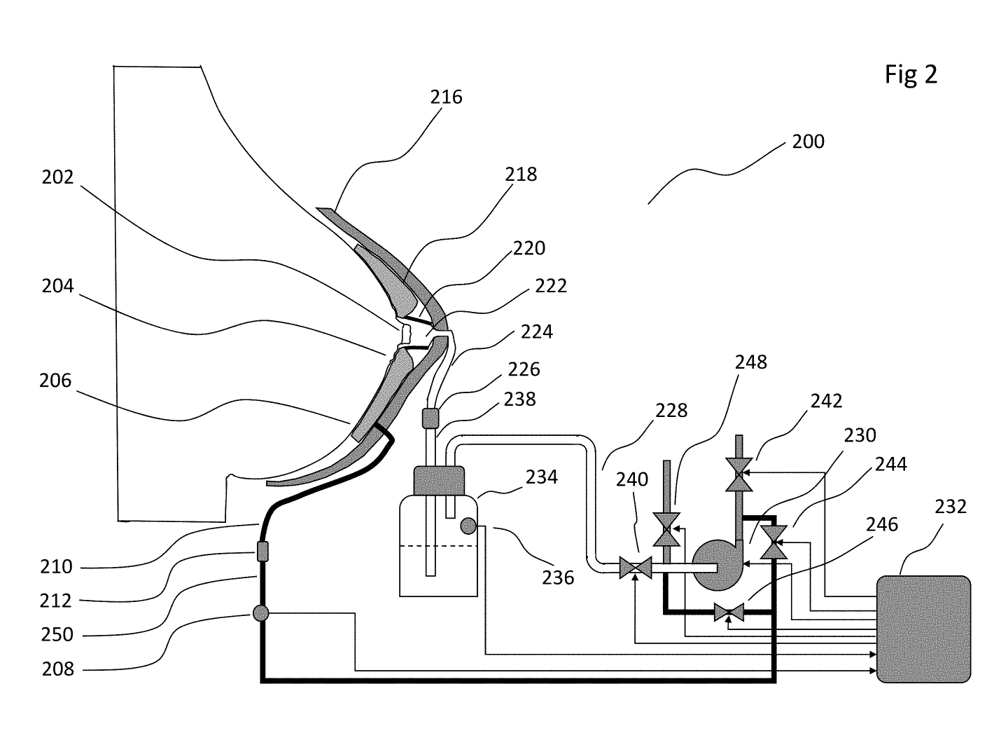

FIG. 2 schematically illustrates a hands-free breast pumping system 200 at the condition of in accordance with one embodiment of the present invention. The pumping system 200 generally comprises a vacuum space 222, air bag 218, and milk hose 224.

In one embodiment, the vacuum space 222 is built by using air bag 218. By inflating air bag 218 using vacuum pump 230, one surface of air bag 218 makes contact with the breast to secure a vacuum in vacuum space 222 effectively. The contact areas include areola 204 and its surrounding area on breast 206.

In one embodiment, only one vacuum pump 230 is used to pump breast milk from vacuum space 222 to milk bottle 234, and also inflate or deflate air bag 218 by controlling on-off states of milk bottle valve 240, out valve 242, bypass valve 244, loop valve 246, and inhale valve 248.

The inflation of air bag 218 is performed by supplying air from vacuum pump 230 to air bag 218 through pump hose 250, air bag hose coupling 212, and air bag hose 210 at the closed condition of loop valve 246 and out valve 242 and the open conditions of bypass valve 244 or inhale vale 248. In one embodiment, the vacuum pump 230 is controlled by control system 232. The control system 232 controls the pressure in air bag 218 based on the measurement resulting from air bag pressure sensor 208 feeding back to control system 232.

For effective contact between air bag 218 and areola 204 and the surrounding area of areola 204 on breast 206, moderate surface area is ensured by design of air bag 218. The materials of air bag 218 plays an important role in effectiveness of seal of vacuum space 222. In one embodiment, the material of air bag 218 is latex rubber. In another embodiment, the material of air bag 218 is silicon rubber, or silicone gel.

To keep a stable vacuum space 222, the connector 220 is configured to keep the contact area in correct position. In one embodiment, one end of the connector 220 makes contact with areola 204 or the side surface of nipple 202. The other end of the connector 220 connects milk hose 224.

FIG. 2 depicts that the air bag 218, connector 220, and milk hose 224 are fixed on bra 216. In one embodiment, air bag 218, connector 220, and milk hose 224 are held by a specially designed strip which is independent of bra 216.

When a baby is sucking milk from the breast, a vacuum and periodical squeeze on nipple are applied. For the simulation of the real sucking of breast milk, in one embodiment, nipple 202 is periodically squeezed by connector 220 by using a pulse pressure in air bag 218 controlled by control system 232. For the purpose of comfort, in one embodiment, the material of connector 120 is silicone gel or silicon rubber.

Breast milk is sucked from nipple 202 through connector 220, milk hose 224, milk hose coupling 226, and bottle hose 238 to milk bottle 234 in the open condition of milk bottle valve 240. The milk pumping strength is determined by the vacuum pressure in milk bottle 234. In one embodiment, vacuum sensor 236 is located in milk bottle 234 for the purpose of control of the vacuum in vacuum space 222. The vacuum pressure data obtained by vacuum sensor 236 is input to control system 232. Control system 232 adjusts pumping air flow rate in air hose 228 by vacuum pump 230 to control the vacuum in milk bottle 234.

In one embodiment, when breast pumping is finished, the air in air bag 218 is pumped out of air bag 218 using vacuum pump 230 at the close conditions of milk bottle valve 240, bypass valve 244, and inhale valve 248, and at the open conditions of loop valve 246 and out valve 242, controlled by control system 232.

In one embodiment, control system 232 comprises a central process unit (CPU), dynamic random-access memory (DRAM), non-volatile memory, analog/digital (A/D) converter, and digital/analog (D/A) converter. Control program is saved in non-volatile memory.

One of the important advantages of this invention is the programmable capability. In one embodiment, the breast pumping schedule is controlled by a program stored in control system 232. This advantage is necessary for breast pump during sleep in the evening when the baby's mother is tired and wishes to have a good night's sleep.

In one embodiment, the breast pump is controlled and programmed by using a cell phone with a pre-programmed mobile app.

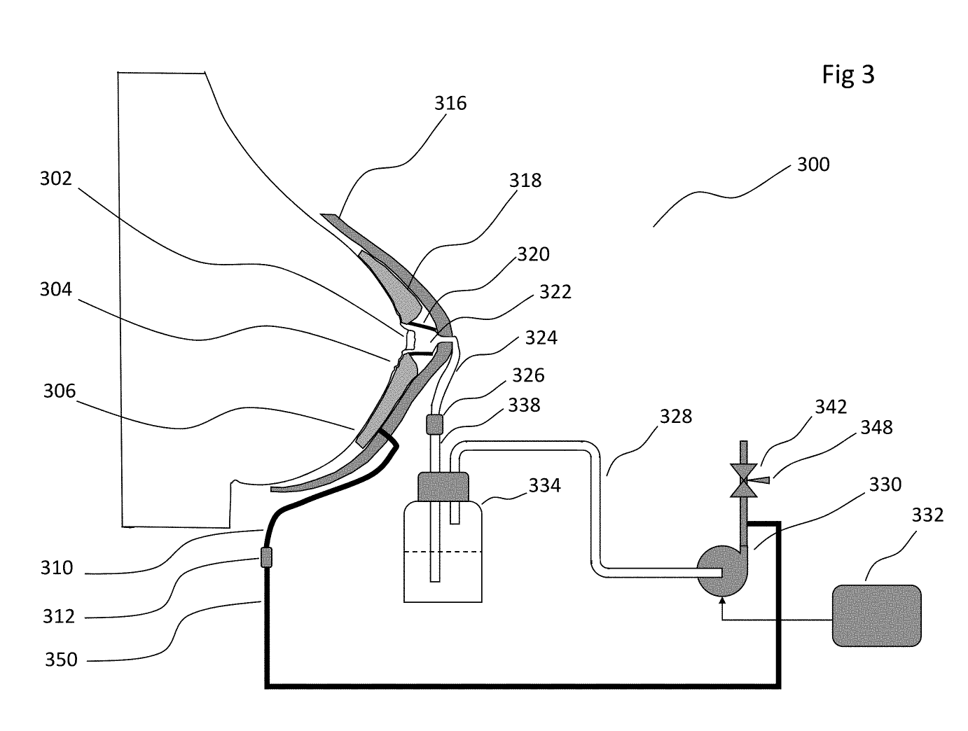

FIG. 3 schematically illustrates a hands-free breast pumping system 300 at the condition of in accordance with one embodiment of the present invention. The pumping system 300 generally comprises a vacuum space 322, air bag 318, and milk hose 324. In one embodiment, air bag 318 has a donut shape for securing a vacuum in vacuum space 322.

The key to pumping breast milk is keeping a moderate vacuum around the nipple area. In one embodiment, the vacuum space 322 is built by using air bag 318. By inflating air bag 318 using vacuum pump 330, one surface of air bag 318 contacts the breast to seal vacuum space 322 effectively. The contact areas include areola 304 and its surrounding area on breast 306.

The inflation of air bag 318 is performed by supplying air from vacuum pump 330 to air bag 318 through pump hose 350, air bag hose coupling 312, and air bag hose 310. In one embodiment, the vacuum pump 330 is controlled by control system 332. In one embodiment, the control system 332 is a switch.

For effective contact between air bag 318 and areola 304 and surrounding area of areola 304 on breast 306, moderate surface area is ensured by the design of air bag 318. The materials of air bag 318 play an important role in effectiveness of seal of vacuum space 322. In one embodiment, the material of air bag 318 is latex rubber. In another embodiment, the material of air bag 318 is silicon rubber, or silicone gel.

To keep stable vacuum space 322, the connector 320 is configured to keep the contact area in its correct position. In one embodiment, one end of the connector 320 contacts areola 304 or side surface of nipple 302. The other end of the connector 320 connects to milk hose 324.

FIG. 3 depicts that the air bag 318, connector 320, and milk hose 324 are fixed on bra 316. In one embodiment, air bag 318, connector 320, and milk hose 324 are held by a specially designed strip which is independent on bra 316.

In one embodiment, the opening of out valve 342 is adjusted by valve adjuster 348. By adjusting the opening of out valve 342, pressure in air bag 318 is adjusted. For this embodiment, the whole system is very simple and thus low-cost.

Breast milk is sucked from nipple 302 through connector 320, milk hose 324, milk hose coupling 326, and bottle hose 338 to milk bottle 334. The milk pumping strength is determined by the vacuum pressure in milk bottle 334. The air in milk bottle 334 is pumped out by vacuum pump 330 via air hose 328.

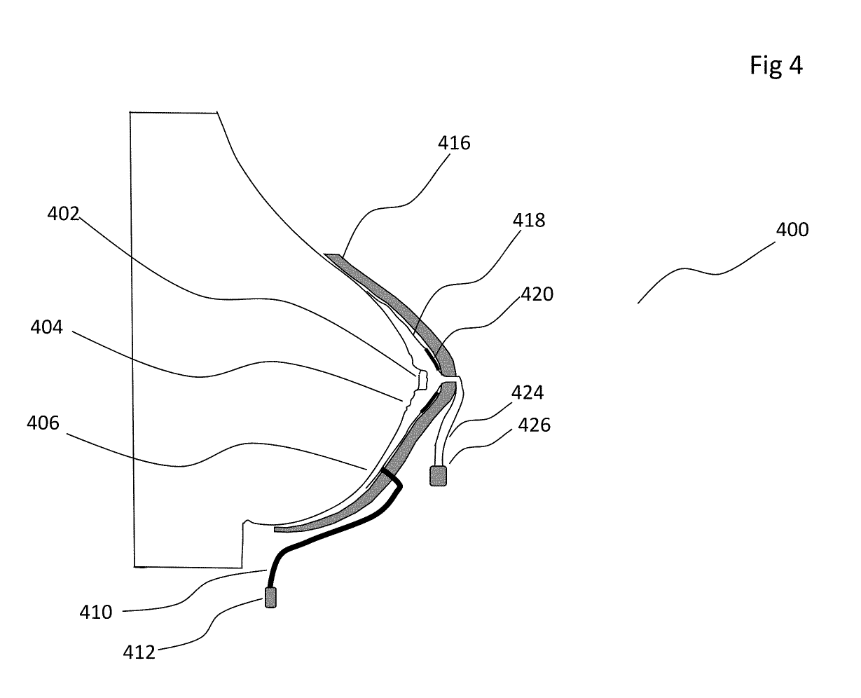

FIG. 4 schematically illustrates the breast area setup 400. The breast area setup 400 includes nipple 402, areola 404, breast 406, air bag hose 410, air bag hose coupling 412, bra 416, air bag 418, connector 420, milk hose 424, and milk hose coupling 426.

In one embodiment, air bag 418 is not inflated when not in use. The milk hose coupling 426 and air bag hose coupling 412 are easily connected to the other part of the pumping system.

In one embodiment, the air bag hose 410, air bag hose coupling 412, milk hose 424, and milk hose coupling 426 are configured to be attached to bra 418 hidden inside a shirt or a coat.

When pumping breast milk, the air bag hose coupling 412 and milk hose coupling 426 are connected to other part of the system without the need to take off the shirt or bra. This configuration makes it very convenient to use.

What is claimed is:

1. A hands-free breast pump system comprising
a. an air bag wherein inflation of said air bag makes one surface of said air bag contact a breast surrounding a nipple, forms an around-nipple space around said nipple;
b. an air hose configured to inflate said air bag;
c. a vacuum pump configured to create vacuum in said around-nipple space for breast milk expression;
d. a milk hose configured to flow breast milk from said around-nipple space to a milk container;

2. The hands-free breast pump system of claim 1 further includes a connector configured to connect said milk hose and said air bag;

3. The hands-free breast pump system of claim 1 further includes an air pump configured to inflate said air bag.

4. The hands-free breast pump system of claim 1, wherein said air bag is inflated by said vacuum pump.

5. The hands-free breast pump system of claim 1, wherein said air bag contacts said areola of said breast, or contact a surrounding area of said areola on said breast, or contact both said areola and said surrounding area of said areola on said breast when said air bag is inflated by said air pump via said air hose.

6. The hands-free breast pump system of claim 1, wherein said around-nipple space comprises a nipple surface, a surface of said connector, and an opening connecting to said milk hose.

7. The hands-free breast pump system of claim 1, wherein said around-nipple space further includes area of said areola.

8. The hands-free breast pump system of claim 1, wherein said around-nipple space builds up vacuum when air is pumped out of said nipple-surround space by said vacuum pump through said milk hose.

9. The hands-free breast pump system of claim 1, wherein said nipple is squeezed periodically by a pressure pulse in said air bag.

10. The hands-free breast pump system of claim 1 further includes a pressure sensor configured to control vacuum pressure in said around-nipple space.

11. The hands-free breast pump system of claim 1 further includes a control system configured to control pumping time and duration, vacuum pressure in said around-nipple space, and pressure in said air bag.
